(19) United States
(12) Patent Application Publication
(10) Pub. No.: US 2011/0020890 A1
(43) Pub. Date: Jan. 27, 2011

(54) Increased ethanol production by bacterial cells

(76) Inventors: Muhammad Javed, London (GB); Brian S. Hartley, London (GB); Namdar Baghaei-Yazdi, London (GB)

Correspondence Address: MCANDREWS HELD & MALLOY, LTD, 500 WEST MADISON STREET, SUITE 3400, CHICAGO, IL, 60661, US

(21) Appl. No.: 12/867,037

(22) PCT Filed: Feb. 12, 2009
(86) PCT No.: PCT/GB2009/000402

§ 371 Date: Aug. 10, 2010

(30) Foreign Application Priority Data
Feb. 13, 2008 (GB) 0802675.9

(51) Int. Cl. C12P 7/06

(52) U.S. Cl. 435/161

(57) ABSTRACT

Fermentation processes for production of ethanol include supplying a thermophilic microorganism lacking lactate dehydrogenase activity with sugars under conditions in which they metabolise them predominantly by the pyruvate-formate lyase pathway. Importantly, the processes also include supplying sufficient glycerol to convert all of the sugars to ethanol. A further embodiment of the invention includes supplying additional glycerol sufficient to convert the exogenous acetate present in biomass hydrolysates into ethanol. Any type of fermentation system can be used for these processes, but a preferred embodiment includes continuous cultures at high temperatures in which ethanol is removed continuously by vacuum evaporation.

### FIELD OF THE INVENTION

This invention relates to enhanced ethanol production by thermophilic bacteria such as Bacilli from mixed sugars, such as those derived from the hydrolysis of biomass. More specifically, it relates to use of waste glycerol from biodiesel production as a co-feedstock to increase ethanol yields in such processes.

### BACKGROUND TO THE INVENTION

Shama, G. and Hartley, B. S. (Phil. Trans. Roy. Soc. Lond. A321, 555-568, 1987) observed that under optimal anaerobic growth conditions a mutant thermophilic Bacillus that lacks lactate dehydrogenase activity (strain LLD-15), like the wild type strain (LLD-R), metabolises a wide range of sugars. However, unlike the wild type strain which predominantly produces lactate as the major product, the mutant strain metabolises these sugars by a pyruvate-formate lyase (PFL) pathway to yield 1 mol. of acetate, 1 mol. of ethanol and 2 mol. of formate per mol. of glucose equivalent consumed (FIG. 1) (San Martin, R., Busshell, D., Leak, D. J. and Hartley, B. S. J. Gen. Microbiol. 139, 1033-1040, (1993)). They also observed that in the mutant strain, under conditions such as low pH and/or high sugar concentrations, cell numbers, formate and acetate concentrations decrease while ethanol concentrations increase with concomitant emergence of pyruvate in the fermentation broth. This suggests that under these conditions, the PFL pathway is suppressed resulting in the accumulation of pyruvate and reducing equivalents in the form of NADH in the cell. Although the increased cellular level of NADH stimulates an anaerobic overflow pathway, the pyruvate dehydrogenase (PDH) pathway which yields 2 mol. of ethanol and 2 mol. of CO₂ per mol. of glucose equivalent consumed (FIG. 1), the extent of the pathway flux is not enough to recycle all of the NADH. Thus, the surplus NADH leads to cellular redox imbalance and cell death, a phenomenon which is defined as ‘Redox Death’. It is also observed that the ‘Redox Death’ phenomenon is more prevalent when the input sugar in the culture is 2% or higher.

Although this allows high yields of ethanol, as required for industrial ethanol production, the non-growing cells in batch or continuous fermentations die or wash away quickly at sugar concentrations greater than around 2% w/v, so such processes are not commercially viable.

The reason for this cell death or ‘Redox Death’ is analysed in International Patent Application Publication Number WO 2007/110608, which proposes a method to avoid Redox Death by using fed-batch fermentations regulated by a variety of sensors so as to control feed rates to maintain sugar concentrations below the ‘critical point’ of 2%. These do indeed allow use of concentrated sugars to produce >4% w/v ethanol, but at the expense of ethanol yield and/or volumetric productivity.

An alternative solution to the problem of ‘Redox Death’ is proposed in International Patent Application Publication Number WO 2007/110606, which describes construction of a new metabolic pathway for the enhancement of ethanol production from other cellular metabolic products.

The above solution is more suitable for converting sugars into ethanol. However, hydrolysates obtained from biomass, in addition to C5+C6 sugars, contain high levels of acetic acid arising from the acetyl groups present in hemicelluloses. The acetic acid can enter the cell and is converted to acetyl CoA. The acetic acid is, therefore, considered an undesirable waste product and is harmful to the growth of the cells by acting as an ‘uncoupling agent’ that reduces membrane potential.

It has been shown that glycerol is a potentially abundant and inexpensive source of reducing equivalents, since it is a low value by-product from conversion of plant triglycerides into biodiesel (S. S. Yazdani and R. Gonsalez, Current Opinions in Biotechnology, 18, 213-219, 2007).

Gonsalez and Yazdani (Patent Application WO 2007115228) have proposed the anaerobic fermentation of glycerol to produce a range of more valuable products such as ethanol by strains of E. coli that have a functional 1,2-propanediol pathway, a functional type II glycerol dehydrogenase-dihydroxyacetone kinase pathway, and a functional F0Fi-ATPase pathway, but lack a functional 1,3-propanediol pathway. The fermentation conditions must be very precise and must contain essential additives such as dihydroxyacetone.

### DESCRIPTION OF THE INVENTION

The pathways proposed in this invention for the anaerobic fermentation of glycerol are completely different to known fermentation processes. They apply to a broad range of thermophiles that lack lactate dehydrogenase (activity) but can metabolise mixed C5 and C6 sugars derived from crude hydrolysates of biomass.

The present invention describes a method for the conversion of acetic acid into ethanol. While the C5+C6 sugars of the hydrolysates can be converted to ethanol and CO₂ (see for example WO 2007/110606), the present invention describes an alternative and complementary route that can provide even higher yields of ethanol from hydrolysates by providing more reducing equivalents (NADH) from a more reduced carbon substrate, such as glycerol and converting acetate to ethanol (FIG. 3).

Accordingly, the invention provides an (industrial) anaerobic fermentation process for production of ethanol comprising supplying at least one thermophilic microorganism lacking lactate dehydrogenase activity with sugars, wherein the at least one thermophilic microorganism is also supplied with glycerol in an amount sufficient to maximise ethanol production whilst minimising acetate production. As discussed herein, the processes of the invention may permit use of exogenous acetate to produce increased levels of ethanol due to the presence of additional NADH produced by the glycerol pathway. The cells may be maintained in redox balance during the fermentations of the invention through supplying sufficient glycerol to minimise acetate production and to maximise ethanol production. In certain embodiments, the fermentation is at least partly carried out under aerobic conditions. As shown in the experimental section below, partially aerobic conditions still result in excellent ethanol yields.

FIG. 2 illustrates the concept of glycerol utilisation for ethanol production. The glycerol is added to anaerobic fermentations of biomass hydrolysates by thermophile cells that lack lactate dehydrogenase activity and are growing at their maximum rate by the pyruvate formate lyase pathway. The glycerol is phosphorylated by ATP using glycerokinase and the glycerol-3-phosphate is oxidised by glycerophosphate deydrogenase to 3-phosphglyceraldehyde. Compared to the sugars glycolytic pathway, this produces an additional reducing equivalent of NADH which can be used to reduce other components in the cell. The phospho-glyceraldehyde is then converted to pyruvate by glycolytic enzymes producing another molecule of NADH. When the PFL pathway flux is saturated, as is the case under the operating conditions of this process, the excess pyruvate will be metabolised by the anaerobic PDH overflow pathway (FIG. 1). However the additional NADH is then available to reduce all of the acetyl-CoA arising from the sugar metabolism to ethanol. The net result is that 1 mole of glucose equivalent+2 mole of glycerol yields 4 moles of ethanol+2 moles of formate+2 moles of CO₂.

Such mixed glucose/glycerol fermentations have obvious advantages over conventional yeast fermentations or even thermophile fermentations, that yield at best 2 moles of ethanol+2 moles of CO₂/mole glucose equivalent. The formate can be used as a cosubstrate for aerobic production of cell inoculums or other fermentations, so the ethanol yield is double and the atmospheric CO₂ released is only half that of yeast fermentations.

Moreover we have seen that the glycerol pathway could also be used to produce ethanol from the exogenous acetate that is an undesirable residue in biomass hydrolysates. Acetate is readily converted by acetyl-CoA synthetase to acetyl-CoA which is then reduced to ethanol in concert with the glycerol pathway which produces additional reducing equivalents (NADH), as illustrated in FIG. 3. The mass balance is that 1 mole of exogenous acetate+2 moles of glycerol yields 3 moles of ethanol+2 moles of CO₂.

Therefore by adjusting the level of glycerol supply (for example by providing additional or extra glycerol) to anaerobic fermentations of biomass hydrolysates it will be possible to convert all of the sugars and the exogenous acetate to ethanol. The only by-products will be formate which can be converted to animal feed in aerobic fermentations and CO₂ (half of that in yeast fermentations). The latter has been shown to be very pure, so will have value for dry ice and soft drinks production.

An additional advantage is that thermophile cells, such as those employed in the present invention, grow very rapidly under high temperature conditions, where concentrated ethanol vapour is readily removed directly from the fermentation under mild vacuum. Therefore the process saves energy by eliminating cooling costs and minimising distillation costs.

The hemicellulosic feedstocks for this process will be derived by mild acid hydrolysis from food processing or agricultural wastes, so will have a minimum carbon footprint. The major products of the processes of the invention would be ethanol and high-protein animal feed, with smaller amounts of atmospheric CO₂ evolution. Hence the bioethanol produced by this process could make a significant contribution to reducing global warming.

Almost any type of fermentation system is compatible with this invention, but it is particularly suitable for continuous cultures, which will be very fast and readily optimised by adjusting the glycerol feed rate to maximise ethanol production and minimise acetate by-product.

Whilst thermophilic microorganisms have lower ethanol tolerance than yeasts (typically below 4% w/v), ethanol production may advantageously be carried out at optimal growth conditions under which ethanol is readily removed through evaporation or distillation. Thus, the fermentation process of the invention may be carried out at a temperature of at least 50° C., preferably at least 70° C. or higher.

In certain embodiments of the invention, ethanol produced in the fermentation is removed continuously so as to reduce ethanol concentration in the fermentation below the ethanol tolerance of the at least one thermophilic microorganism. Ethanol produced during the fermentation process may be continuously and conveniently removed from the high temperature fermentation by membrane and/or (mild) vacuum evaporation in specific embodiments. This will reduce the process cost and energy required to produce 95% w/v ethanol for biofuel formulations.

Any suitable thermophilic microorganism may be utilised in the processes of the invention, including the specific thermophilic microorganisms described herein. In one embodiment, the at least one thermophilic microorganism is of the genus Bacillus and preferably comprises Bacillus stearothermophilus. In a specific preferred embodiment, the Bacillus is a derivative of Bacillus stearothermophilus strain LLD-R(NCIMB 12403) or strain LLD-15 (NCIMB 12428). In a further embodiment, the thermophilic microorganism is Geobacillus thermoglucosidasius.

As stated above, the thermophilic microorganism used in the fermentation processes of the invention lacks lactate dehydrogenase activity. This may be achieved through any suitable means. In one embodiment, the at least one thermophilic microorganism lacks lactate dehydrogenase activity due to inactivation of the gene encoding lactate dehydrogenase (ldh gene). Gene inactivation may be achieved through any suitable route.

In one embodiment, the at least one thermophilic microorganism lacks lactate dehydrogenase activity due to transformation with a DNA construct comprising a nucleotide sequence encoding a non-functional lactate dehydrogenase, wherein the nucleotide sequence encoding a non-functional lactate dehydrogenase leads to inactivation of lactate dehydrogenase activity through recombination with the gene encoding lactate dehydrogenase in the genome of the thermophilic microorganism. Such DNA constructs are known in the art and described for example in WO 2007/110606, incorporated by reference herein.

The invention will now be described with reference to the following non-limiting description and figures.

### BRIEF DESCRIPTION OF THE FIGURES

FIG. 1 summarises the glycolytic pathway and the pyruvate-formate lyase pathway which produces 2 moles of acetyl-CoA and 2 moles of formate from each mole of glucose consumed and is the major growth pathway in thermophile strains that lack lactate dehydrogenase activity. The NADH produced through the glycolytic pathway is only sufficient to reduce one mole of acetyl-CoA to produce ethanol, so to maintain redox balance the other is converted to acetate via acetyl-phosphate with the production of ATP. At high sugar concentrations, this pathway becomes saturated and the excess pyruvate produced by unregulated glycolysis is metabolised via the pyruvate dehydrogenase pathway and reduced by the excess NADH to produce 2 ethanol+2 CO₂.

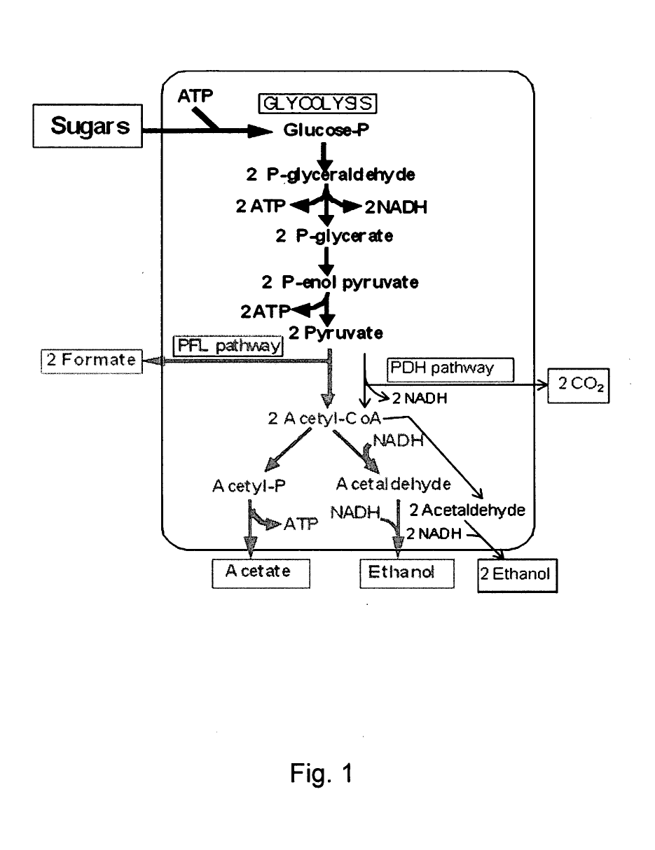

FIG. 2 shows the effect of feeding glycerol, according to the invention, to strains that are growing on sugars under conditions that favour the PFL-pathway. The glycerol is phosphorylated by ATP using glycerokinase and the glycerol-3-phosphate is then oxidised by glycerophosphate deydrogenase to 3-phosphglyceraldehyde. This produces an additional equivalent of NADH which can be used to reduce other components in the cell. The phospho-glyceraldehyde is then converted to ethanol by glycolysis and the PFL pathway. However the additional NADH is then sufficient to reduce all of the acetyl-CoA arising from the sugar metabolism to ethanol. The net result is that 1 mole of glucose equivalent+2 moles of glycerol yields 4 moles of ethanol+2 moles of formate+2 moles of CO₂.

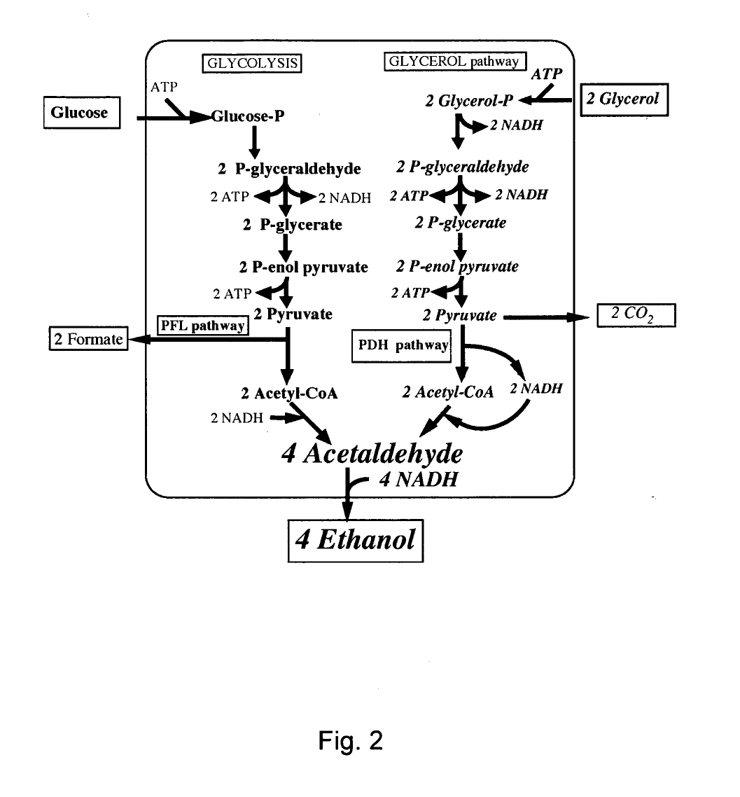

FIG. 3 shows that exogenous acetate is converted to acetyl-CoA and can then be reduced to ethanol by the excess NADH that arises from the glycerol pathway, according to the invention, and the overflow pyruvate dehydrogenase pathway. Hence 3 extra moles of ethanol will arise from 1 mole of acetate and 2 moles of glycerol.

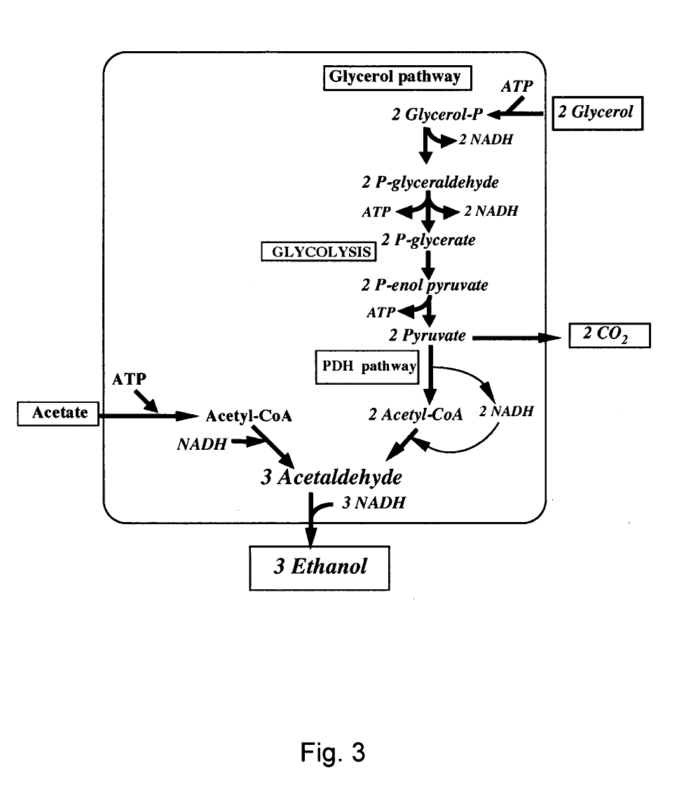

FIG. 4 shows product formation by the BCT25-H strain in 30 ml Sterilin bottles containing 10 ml 2TY medium with 56 mM glucose and different concentrations of glycerol at 65° C. and 200 rpm under partial aerobic conditions.

### EXAMPLE

BCT25-H strain, which is a lactate dehydrogenase-deficient strain of Bacillus derived from strain LLD-R (constructed according to Example 3 of WO 2007/110606), was grown in 30 ml Sterilin bottles containing 10 ml of 2TY medium (tryptone 16 g, yeast extract 10 g, sodium chloride 5 g, and distilled water to 1000 ml. pH 7.0 adjusted with 20% w/v NaOH) with 56 mM of glucose and containing different concentrations of glycerol (54, 108 and 216 mM) at 65° C. and 200 rpm. The experiment was conducted such that the growth conditions were partially aerobic.

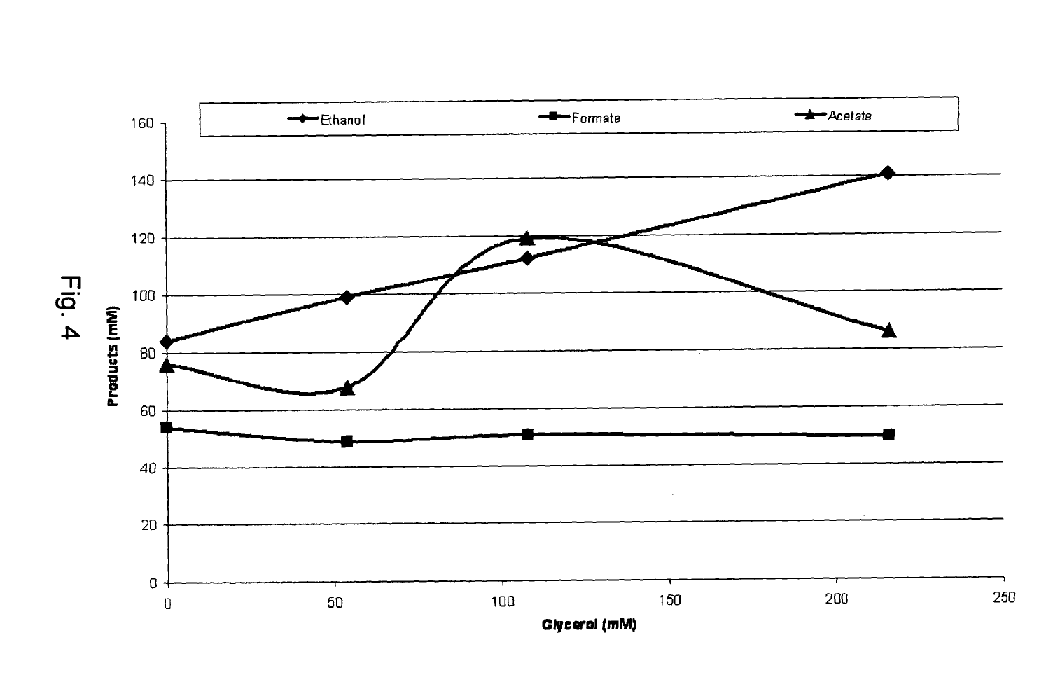

The growth studies showed that the addition of glycerol significantly improved the ethanol production while formate levels did not vary much (see table 1 and FIG. 4). Although a lesser amount of acetate was expected by the addition of glycerol, the slightly higher acetate levels obtained in this study are possibly due to the fact that the growth conditions were not strictly anaerobic and some of the acetate had been produced by the overflow metabolism.

TABLE 1
Summary of ethanol, formate and acetate production (mM)
at varying concentrations of glycerol (mM) in the feed.

| Glycerol | Ethanol | Formate | Aceate |
|---|---|---|---|
| 0 | 84 | 54 | 76 |
| 54 | 99 | 49 | 68 |
| 108 | 112 | 51 | 119 |
| 216 | 141 | 50 | 86 |

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

Various publications are cited herein, the disclosures of which are incorporated by reference in their entireties.

1. A fermentation process, for production of ethanol comprising supplying at least one thermophilic microorganism lacking lactate dehydrogenase activity with sugars, wherein the at least one thermophilic microorganism is maintained in redox balance through supplying sufficient glycerol to minimise acetate production and to maximise ethanol production.

2. The process of claim 1 which is performed under partially aerobic conditions.

3. The process of claim 1 wherein the sugars are supplied at concentrations of between approximately 1% and 30%.

4. The process of claim 1 wherein the sugars are mixed sugars.

5. The process of claim 4 wherein the sugars are derived from the hydrolysis of biomass.

6. The process of claim 1 wherein extra glycerol is supplied to produce ethanol from by-products.

7. The process of claim 1 wherein the glycerol is supplied as a by-product of a process.

8. The process of claim 1 which is carried out in continuous culture with continuous feeds of sugars and glycerol regulated to maximise ethanol production and minimise acetate production.

9. The process of claim 1 wherein the process is carried out in batch or fed-batch culture with continuous or intermittent supply of glycerol regulated to maximise ethanol production and minimise acetate production.

10. The process of claim 1 which is carried out at temperatures exceeding 50° C.

11. The process of claim 1 wherein ethanol is removed continuously by vacuum evaporation or membrane pervaporation.

12. The process of claim 1 wherein the at least one thermophilic microorganism is of the genus Bacillus.

13. The process of claim 12 wherein the Bacillus is Bacillus stearothermophilus or Geobacillus thermoglucosidasius.

14. The process of claim 13 wherein the Bacillus is a derivative of Bacillus stearothermophilus strain LLD-R(NCIMB 12403) or strain LLD-15 (NCIMB 12428).

15. The process of claim 1 wherein the at least one thermophilic microorganism lacks lactate dehydrogenase activity due to inactivation of a gene encoding lactate dehydrogenase.

16. The process of claim 1, wherein the fermentation process is an anaerobic fermentation process. (From claim 1)

17. The process of claim 2, wherein the sugars are supplied at concentrations of between approximately 1% and 30%. (From claim 3)

18. The process of claim 4, wherein the mixed sugars include pentose sugars.

19. The process of claim 6, wherein the by-products is acetate endogenously produced by the microorganism, or exogenously present acetate in the biomass hydrolysates.

20. The process of claim 7, wherein the process producing the by-products is biodiesel production.
